(19) United States
(12) Patent Application Publication
(10) Pub. No.: US 2010/0256394 A1
(43) Pub. Date: Oct. 7, 2010

(54) METHOD FOR THE PREPARATION OF LASOFOXIFENE

(76) Inventors: Petr Lustig, Pardubice (CZ); Ludmila Hejtmankova, Prelouc (CZ)

Correspondence Address: Pearl Cohen Zedek Latzer, LLP, 1500 Broadway, 12th Floor, New York, NY, 10036, US

(21) Appl. No.: 12/602,152

(22) PCT Filed: May 28, 2008
(86) PCT No.: PCT/CZ2008/000058

§ 371 Date: Jun. 8, 2010

(30) Foreign Application Priority Data
May 29, 2007 (CZ) PV 2007-373

(51) Int. Cl. C07D 207/06

(52) U.S. Cl. 548/576

(57) ABSTRACT

A method of preparing (−)-cis-(5R,6S)-6-phenyl-5-[4-(2-pyrrolidin-1-ylethoxy)phenyl]-5,6,7,8-tetrahydronaphthalen-2-ol D-tartrate-lasofoxifene of formula 1, comprising the following steps a) Preparation of cis-1-{2-[4-(2-phenyl-6-methoxy-1,2,3,4-tetrahydronaphthalen-1-yl)-phenoxy]ethyl}pyrrolidine of formula (3) by alkylation of cis-1-(4-hydroxyphenyl)-2-phenyl-6-methoxy-1,2,3,4-tetrahydronaphthalene with 1-(2-chloroethyl)pyrrolidine base or its salt, b) Deprotection of the hydroxyl group in the substance of formula (3) by the effect of hydrobromic acid generating cis-6-phenyl-5-[4-(2-pyrrolidin-1-ylethoxy)phenyl]-5,6,7,8-tetrahydronaphthalen-2-ol hydrobromide of formula (2a), c) Conversion of the substance of formula (2a) into cis-6-phenyl-5-[4-(2-pyrrolidin-1-ylethoxy)phenyl]-5,6,7,8-tetrahydronaphthalen-2-ol of formula (2b), d) Preparation of lasofoxifene of formula (1) by conversion into the corresponding diasteroisomer by reaction with D-tartaric acid and crystallization.

### TECHNICAL FIELD

The invention deals with a method of preparation of highly pure (−)-cis-(5R,6S)-6-phenyl-5-[4-(2-pyrrolidin-1-ylethoxy)phenyl]-5,6,7,8-tetrahydronaphthalen-2-ol, or 2-naphthalenol, 5,6,7,8-tetahydro-6-phenyl-5-[4-[2-(1-pyrrolidinyl)ethoxy]phenyl]-(5R,6S) known under the name lasofoxifene.

### BACKGROUND ART

Lasofoxifene is an oestrogen antagonist in the bones and it is useful for applications like oral contraception, relief from climacteric symptoms, prevention of a threatening or habitual abortion, relief from dysfunctional uterine bleeding, mitigation of endometriosis, support of ovary development, treatment of acne, reducing excessive hair growth in women, prevention and treatment of cardiovascular diseases, atherosclerosis, osteoporosis, treatment of benign hyperplasia of prostate and prostate carcinoma, treatment of obesity. This compound also manifests a favourable effect onto the level of lipids in blood plasma and as such it is useful for treatment and prevention of hypercholesterolemia. Lasofoxifene also acts as an anti-oestrogen in breast tissue and this is why it is useful for treatment and prevention of breast cancer. It is used as the free base or D-tartaric salt.

6-Phenyl-5-[4-(2-pyrrolidin-1-yl-ethoxy)phenyl]-5,6,7,8-tetrahydronaphthalen-2-ol has two asymmetric carbon atoms in its molecule and it may exist in four enantiomeric forms. The pharmacologically active form is the 5R,6S isomer (−)-cis-6-phenyl-5-[4-(2-pyrrolidin-1-yl-ethoxy)phenyl]-5,6,7,8-tetrahydronaphthalen-2-ol, lasofoxifene of formula 1.

Lasofoxifene is prepared with several methods. U.S. Pat. No. 3,274,213 (1966) describes synthesis of 1-[2-[4-(6-methoxy-2-phenyl-3,4-dihydronaphthalen-1-yl)phenoxy]ethyl]-pyrrolidine hydrochloride, Nafoxidene hydrochloride 4, as the key intermediate for the preparation of lasofoxifene starting from 3-methoxyacetophenone and 4-(2-pyrrolidinoethoxy)bromobenzene 13, see Scheme 1 below.

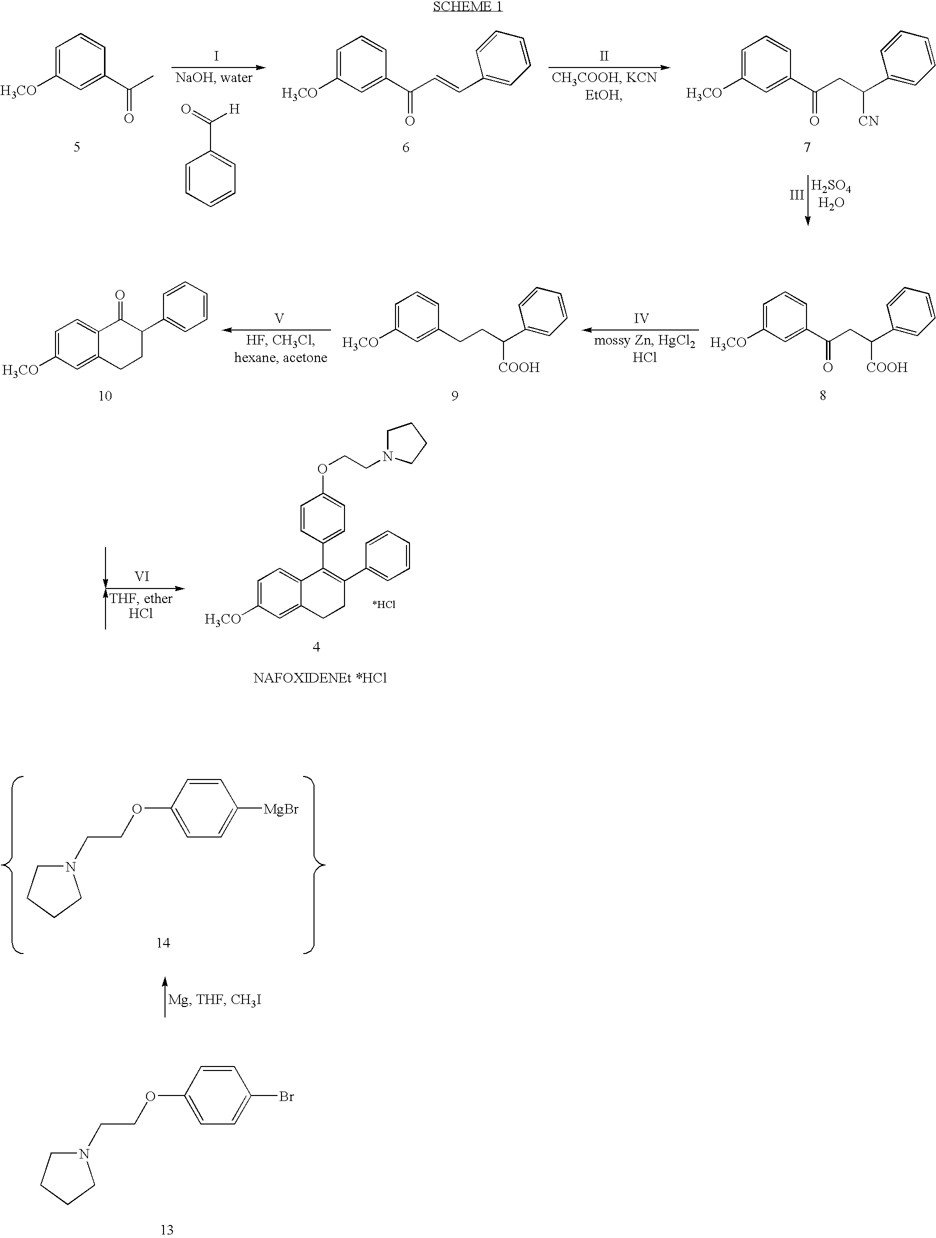

The more recent patent no. EP 0802910 (1995) describes a method of preparation of 1-[2-[4-(6-methoxy-2-phenyl-3,4-dihydronaphthalen-1-yl)phenoxy]ethyl]pyrrolidine hydrochloride, Nafoxidene hydrochloride 4, starting from 6-methoxy-1-tetralone and 4-(2-pyrrolidinoethoxy)-bromobenzene 13, see Scheme 2.

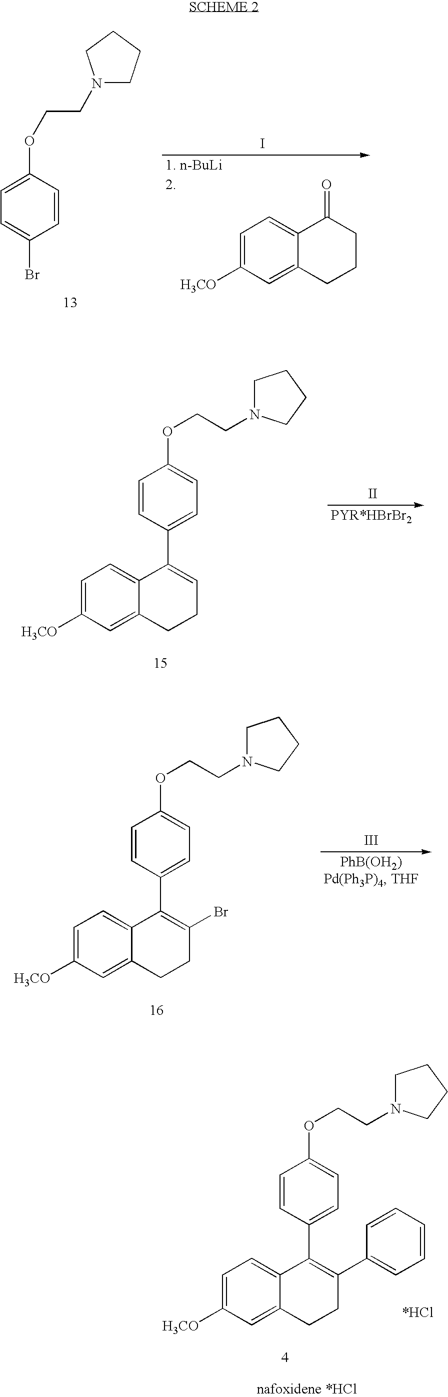

According to patent no. EP 1055658 (2000) cis-1-[2-[4-(6-methoxy-2-phenyl-1,2,3,4-tetrahydronaphthalen-1-yl)phenoxy]ethyl]pyrrolidine 3 is prepared by a 6-stage synthesis starting from 2-bromo-5-methoxytoluene 17 and 4-benzyloxybenzonitrile 24, see Scheme 3.

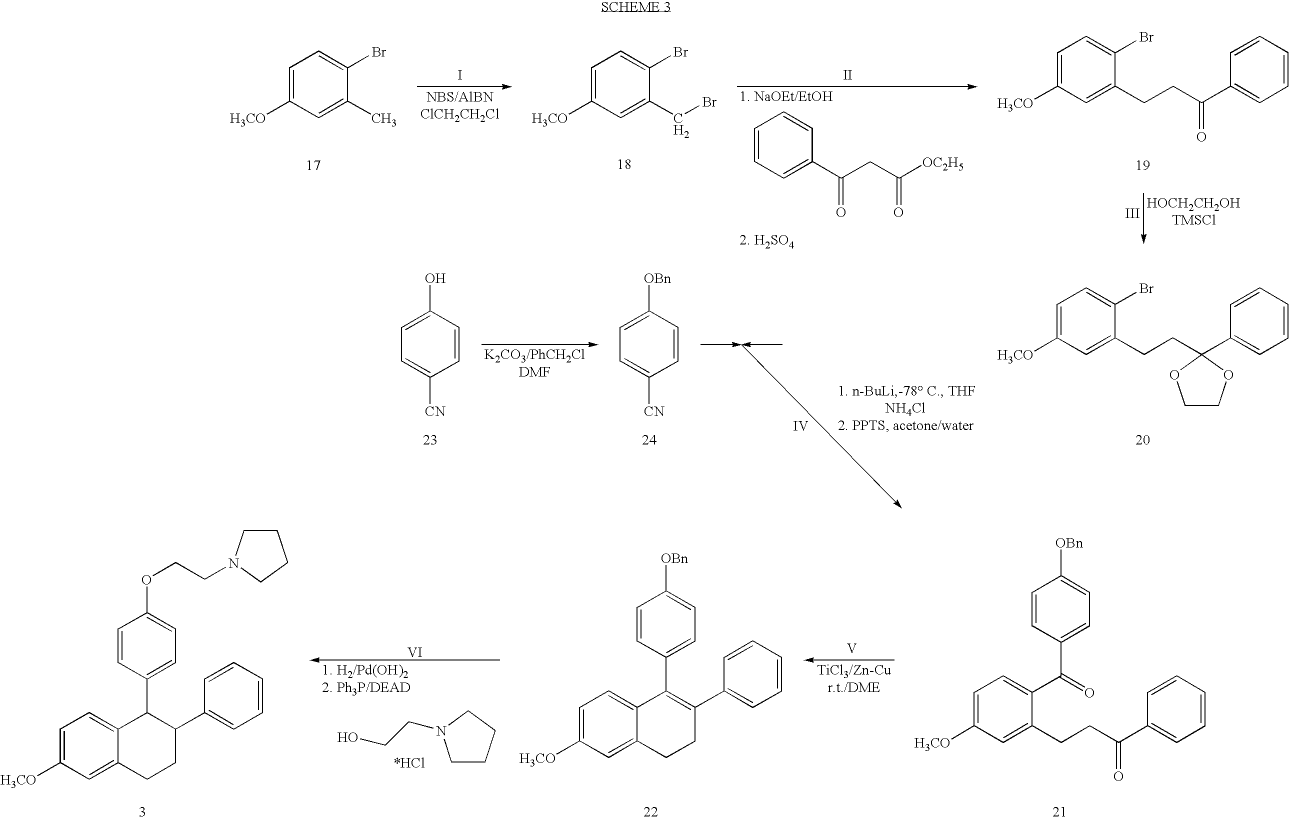

Substance 3, cis-1-{2-[4-(6-methoxy-2-phenyl-1,2,3,4-tetrahydronaphthalen-1-yl)phenoxy]ethyl}pyrrolidine, is prepared by reduction of the double bond in position 5,6 of nafoxidene hydrochloride 4. The reduction performed in a hydrogenating way results in a mixture of cis diastereisomers, see Scheme 4.

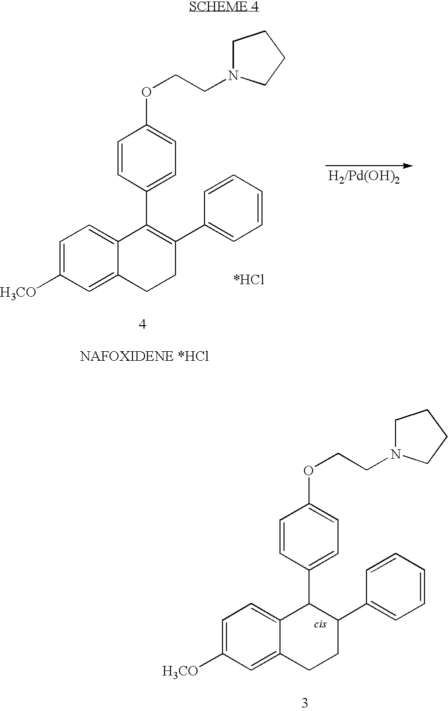

According to patent no. EP 0802910 the reduction is achieved through hydrogenation in absolute ethanol in the presence of palladium hydroxide on active coal as a catalyst under the pressure of 343.5 kPa at the temperature of 20° C. for 19 hours.

Deprotection of the methoxy group in position 6 of intermediate 3, see Scheme 5, is performed in two similar ways described in patent no. EP 0802910. The former is carried out in dichloromethane by the action of boron bromide. The latter consists in deprotection by the action of hydrobromic acid in acetic acid.

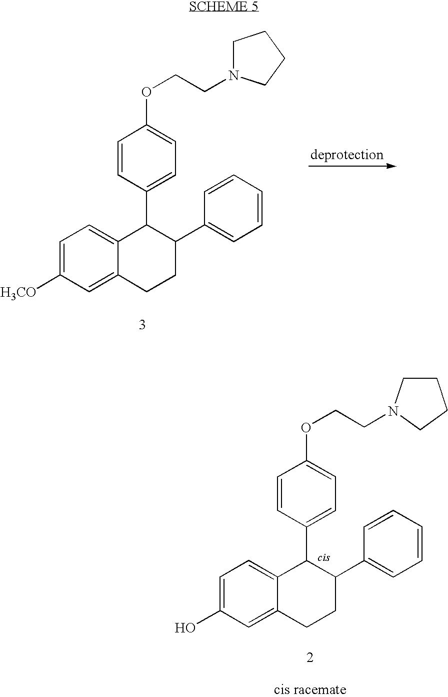

Optical resolution and at the same time purification of the racemate 2, see Scheme 6, was formerly achieved through crystallization of R-(−)-1,1′-binaphthyl-2,2′-dihydrogen-phosphate salt (R-binap), as described in patent no. EP 0802910. The achieved optical purity is 98%. As R-binap is not a pharmaceutically suitable salt, it is necessary to convert the R-binap product into the free base and this free base into a pharmaceutically suitable salt.

A newer method of optical resolution described in the patent no. WO 9716434 (1996) is based on dissolution of racemic lasofoxifene 2 in boiling 95% ethanol with the equimolar quantity of D-tartaric acid. After cooling to the room temperature the desired cis (−) isomer is separated in the form of a solid substance. This product manifests ca. 95% optical purity.

Literature also describes methods of optical purification by enzymatic route in patent no. EP 1156120 or by means of microbial cultures in patent no. EP 0989187.

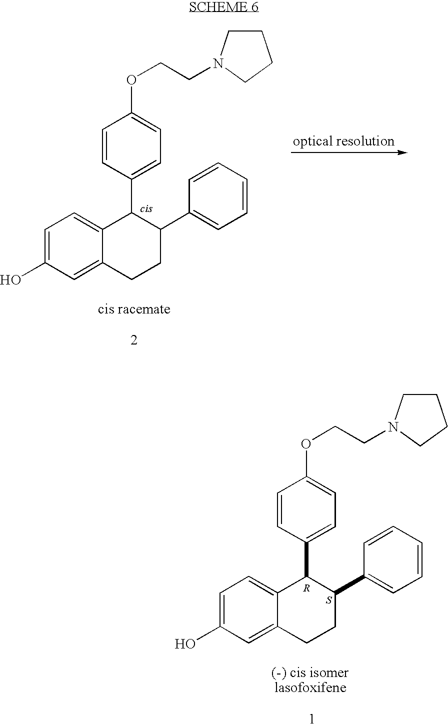

A problem of preparation of (−)-cis-(5R,6S)-6-phenyl-5-[4-(2-pyrrolidin-1-ylethoxy)phenyl]-5,6,7,8-tetrahydronaphthalen-2-ol includes formation of a considerable quantity of poorly identifiable impurities. According to classical arrangements of preparation of racemic lasofoxifene described in the literature the raw product is obtained in the base form, which cannot be resolved with D-tartaric acid in order to obtain the desired optical isomer in the crystalline form in a quality required for an active pharmaceutical ingredient.

### DISCLOSURE OF INVENTION

The invention provides a method of preparation of highly pure (−)-cis-(5R,6S)-6-phenyl-5-[4-(2-pyrrolidin-1-ylethoxy)phenyl]-5,6,7,8-tetrahydronaphthalen-2-ol (lasofoxifene), which is based on obtaining racemic lasofoxifene hydrobromide 2a in a way that minimizes the generation of impurities in individual synthetic stages. The salt obtained this way can be further efficiently purified with crystallization. Crystalline lasofoxifene hydrobromide with a content of the main substance of more than 97% according to HPLC is fully characterized by X-ray, DSC and ¹³C CP-MAS NMR. The high-quality racemic base released from the highly pure hydrobromide is further re-purified by crystallization and this way a crystalline substance characterized by X-ray, DSC and ¹³C CP-MAS NMR can be obtained, contrary to the solidified foam prepared in the literature that cannot be finally purified by crystallization.

### DETAILED DESCRIPTION OF THE INVENTION

Experiments have shown that the conventional method of synthesis of cis-6-phenyl-5-[4-(2-pyrrolidin-1-ylethoxy)phenyl]-5,6,7,8-tetrahydronaphthalen-2-ol (cis-racemate of lasofoxifene) in accordance with patent application no. WO 9621656 produces a product with impurities isolated in the form of a non-crystalline base that does not provide, in subsequent reactions, (−)-cis-(5R,6S)-6-phenyl-5-[4-(2-pyrrolidin-1-ylethoxy)phenyl]-5,6,7,8-tetrahydro-naphthalen-2-ol tartrate in a solid crystalline form in the quality required for API.

### Preparation of cis-1-{2-[4-(2-phenyl-6-methoxy-1,2,3,4-tetrahydronaphthalen-1-yl)phenoxy]ethyl}pyrrolidine (3)

Alkylation of cis-1-(4-hydroxyfenyl)-2-phenyl-6-methoxy-1,2,3,4-tetrahydro-naphthalene with 1-(2-chloroethyl)pyrrolidine base or its hydrochloride in common configuration such as DMF/K₂CO₃, CH₃OH/CH₃O⁻, DMSO/potassium t-butoxide or DMF/NaH did not occur at all or just partly. The isolated products contained side substances. Surprisingly, it has been proved that the reaction has a virtually quantitative character if it is carried out in a specially dried acetone with freshly calcined potassium carbonate under reflux conditions. Although the reaction time of 24 h is relatively long, the reaction mixture does not contain any side substances and the isolation of the product is easy. The inorganic base can be directly removed by filtration, the solvent can be removed by distillation and in order to remove of the salt residues the ethyl acetate solution can be shaken with water. After the removal of the solvent the product is obtained in the form of colourless oil.

### Preparation of cis-6-phenyl-5-[4-(2-pyrrolidin-1-ylethoxy)phenyl]-5,6,7,8-tetrahydronaphthalen-2-ol hydrobromide (2a)

Deprotection of the hydroxy group in position 6 of cis-1-{2-[4-(2-phenyl-6-methoxy-1,2,3,4-tetrahydronaphthalen-1-yl)-phenoxy]ethyl}pyrrolidine 3 as described in patent no. EP 0802910 both by the action of boron bromide and by the action of hydrobromic acid in acetic acid provides the raw product in the base form as greyish-green foam that solidifies, but cannot be purified by crystallization. The quality of the product is not sufficient and resolution with D-tartaric acid does not provide a crystalline substance.

We have tried to perform the deprotection in hydrobromic acid only, under reflux conditions in an inert atmosphere. To our surprise the reaction time was reduced to about a half, the reaction mixture contained considerably fewer dark impurities and after cooling slightly pinkish lasofoxifene hydrobromide was separated in the reaction mixture. The reaction mixture was then poured onto ice and stirred until its dissolution. The solid product was filtered off and washed with water until neutral reaction. In this way, the hydrobromide of cis-racemate of lasofoxifene 2a, which has not been isolated or described in the literature so far, was obtained. In the subsequent experiments we managed to find a method of purification of the raw hydrobromide by crystallization from solvents from the group of alcohols and to characterize the crystalline form of the hydrobromide by means of X-ray and DSC. This product quality was sufficient for performing the optical resolution of the released base.

### Preparation of the free base of cis-6-phenyl-5-[4-(2-pyrrolidin-1-ylethoxy)phenyl]-5,6,7,8-tetrahydronaphthalen-2-ol (2b)

The free base described in the patents was always prepared by evaporation of the solvent from the free base solution. The product had the form of more or less colourless solidified foam. The authors of patent application no. WO 03/082814 tried to purify it by stirring the suspension in a solvent mixture of ethanol and tetrahydrofuran under various temperatures and for various time periods. Using this method we have not always managed to achieve the desired quality of the racemic base. The array of impurities largely depends on the selected preparation method of API. If the base is prepared from the crystalline hydrobromide with purity higher than 97% in accordance with HPLC, we have proved that the substance can be prepared in the crystalline form. The base crystallizes in the form of a solvate with diethyl ether from a solvent mixture of methanol and diethyl ether in various proportions with the formation of polymorph I. Through long-term drying in a vacuum drier at the temperature of 60° C. diethyl ether can be removed from the crystal lattice and desolvated polymorph II can be prepared. Highly pure bases can be crystallized from alcoholic solvents as shown in the following examples.

### BRIEF DESCRIPTION OF DRAWINGS

FIG. 1 shows X-ray powder diffraction of the crystalline salt of cis-6-phenyl-5-[4-(2-pyrrolidin-1-ylethoxy)phenyl]-5,6,7,8-tetrahydronaphthalen-2-ol hydrobromide (2a) in accordance with Example 2.

FIG. 2 shows X-ray powder diffraction of the crystalline form of lasofoxifene base polymorph I prepared in accordance with Example 4.

FIG. 3 shows X-ray powder diffraction of the crystalline form of lasofoxifene base polymorph II prepared in accordance with Example 4.

FIG. 4 shows X-ray powder diffraction of the crystalline form of lasofoxifene base polymorph II prepared by crystallization from ethanol in accordance with Example 5.

FIG. 5 shows X-ray powder diffraction of the crystalline form of lasofoxifene base polymorph II prepared by crystallization from acetone in accordance with Example 6.

FIG. 6 represents FTIR records of the crystalline forms of lasofoxifene hydrobromide and lasofoxifene base polymorph I prepared in accordance with Examples 2 and 4. A Nicolet Nexus (Termo, USA) FTIR spectrometer was used in the ATR configuration, accumulation of 64 scans, resolution 2 l/cm.

FIG. 7 represents records of Raman spectra of the crystalline forms of lasofoxifene hydrobromide and lasofoxifene base polymorph I prepared in accordance with Examples 2 and 4.

FIG. 8 shows the DSC curve of the crystalline salt of cis-6-phenyl-5-[4-(2-pyrrolidin-1-ylethoxy)phenyl]-5,6,7,8-tetrahydronaphthalen-2-ol hydrobromide (2a) in accordance with Example 2.

FIG. 9 shows the DSC curve of the crystalline form of lasofoxifene base polymorph I prepared in accordance with Example 4.

FIG. 10 represents ¹³C CP-MAS NMR of the crystalline salt of cis-6-phenyl-5-[4-(2-pyrrolidin-1-ylethoxy)phenyl]-5,6,7,8-tetrahydronaphthalen-2-ol hydrobromide (2a) in accordance with Example 2.

FIG. 11 represents ¹³C CP-MAS NMR of the crystalline form of lasofoxifene base polymorph I prepared in accordance with Example 4.

FIG. 12 represents ¹³C CP-MAS NMR of the crystalline form of lasofoxifene base polymorph II prepared in accordance with Example 4.

The diffraction patterns were measured with the use of an X'PERT PRO MPD PANalytical diffractometer under the following experimental conditions:
Radiation: CuKα (∥=1.5402 Å)
Monochromator: graphite
Excitation voltage: 45 kV
Anodic current: 40 mA
Measured range: 4-40° 2θ
Increment: 0,008 2θ
Flat sample with the area/thickness of 10/0.5 mm

### EXAMPLES

### Example 1

### Preparation of cis-1-{2-[4-(2-phenyl-6-methoxy-1,2,3,4-tetrahydronaphthalen-1-yl)phenoxy]ethyl}pyrrolidine (3)

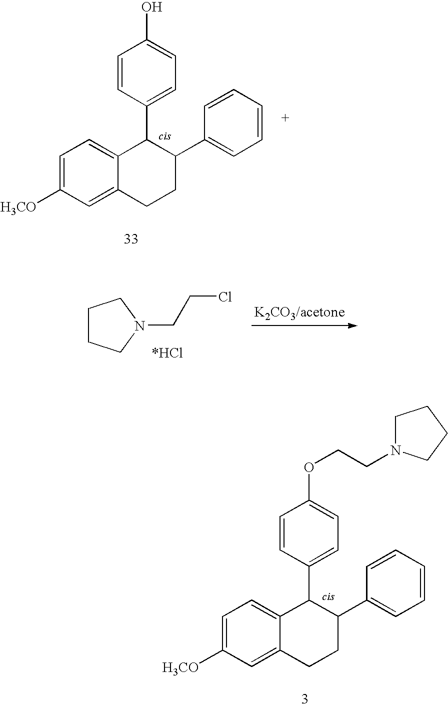

Phenol 33 14.86 g and 1-(2-chloroethyl)pyrrolidine hydroxide 11.5 g are dissolved in 1000 ml of dry acetone. The amount of 68 g of K₂CO₃ dried by annealing is added and the mixture is refluxed while being stirred for 20 hours. After the end of the reaction the salts are filtered off and the solvent is evaporated under reduced pressure. The evaporation residue is dissolved in ethyl acetate and extracted with water twice. The ethyl acetate solution is dried and the solvent is removed by distillation. A waxy light yellow product 3 is obtained in the base form.

### Example 2

### Preparation of cis-6-phenyl-5-[4-(2-pyrrolidin-1-ylethoxy)phenyl]-5,6,7,8-tetrahydronaphthalen-2-ol hydrobromide (2a)

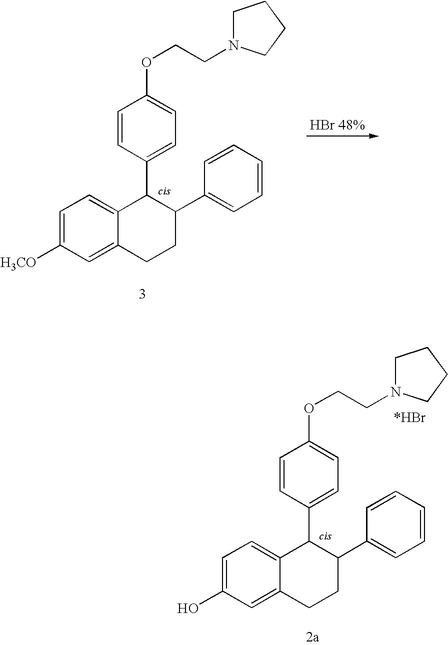

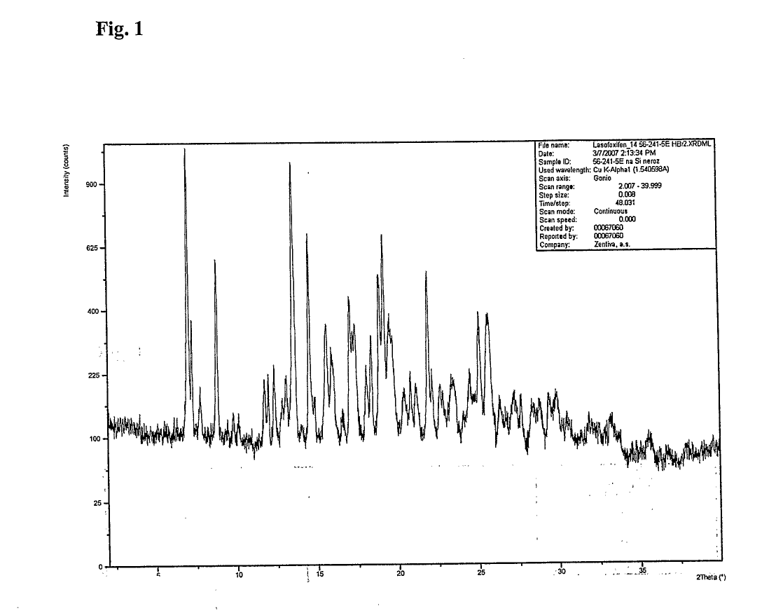

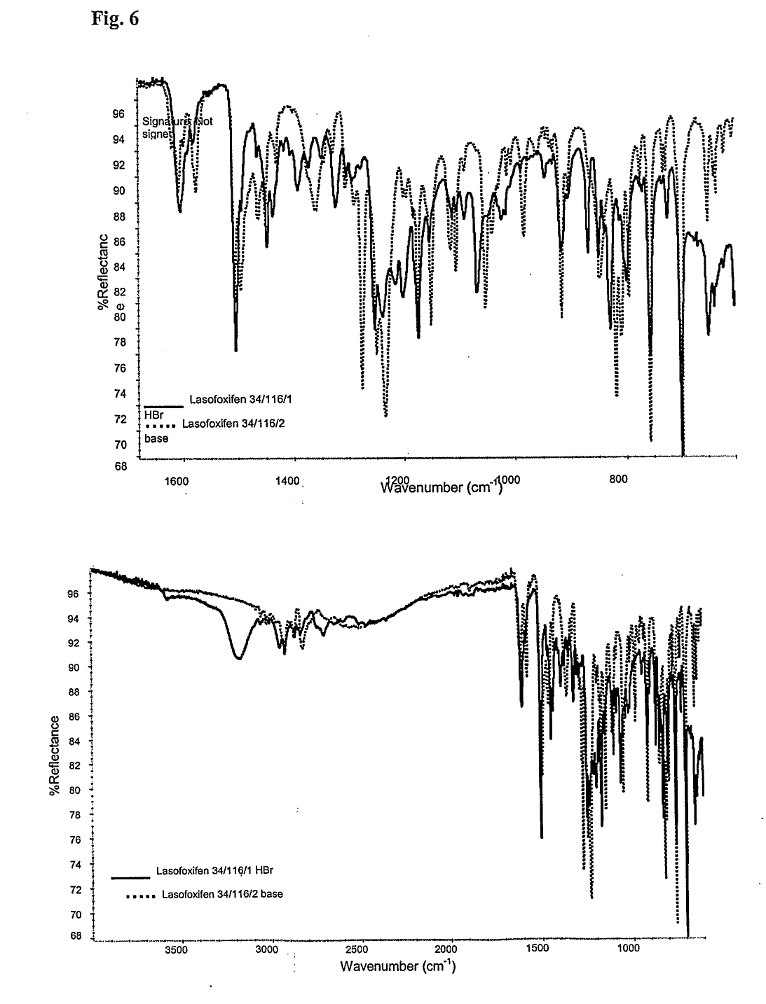

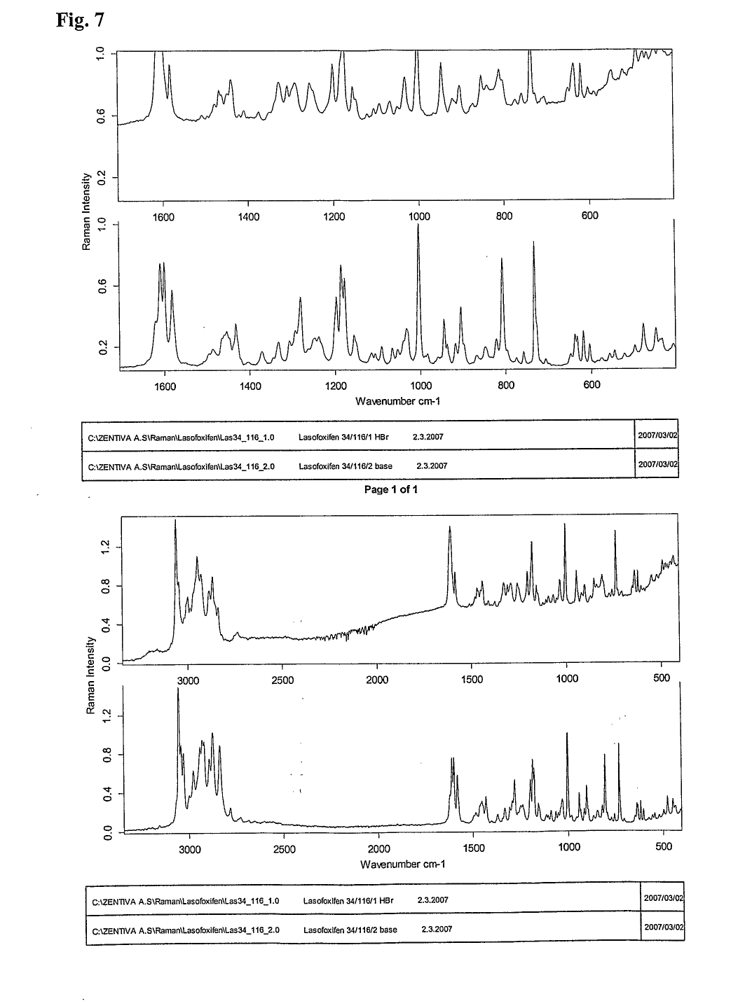

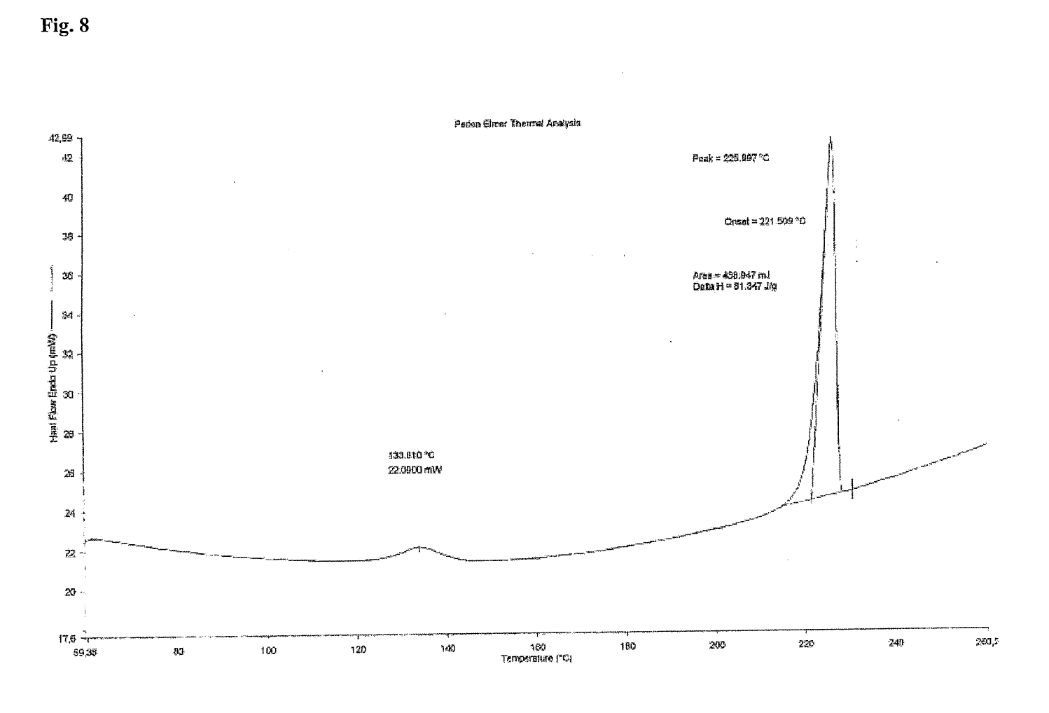

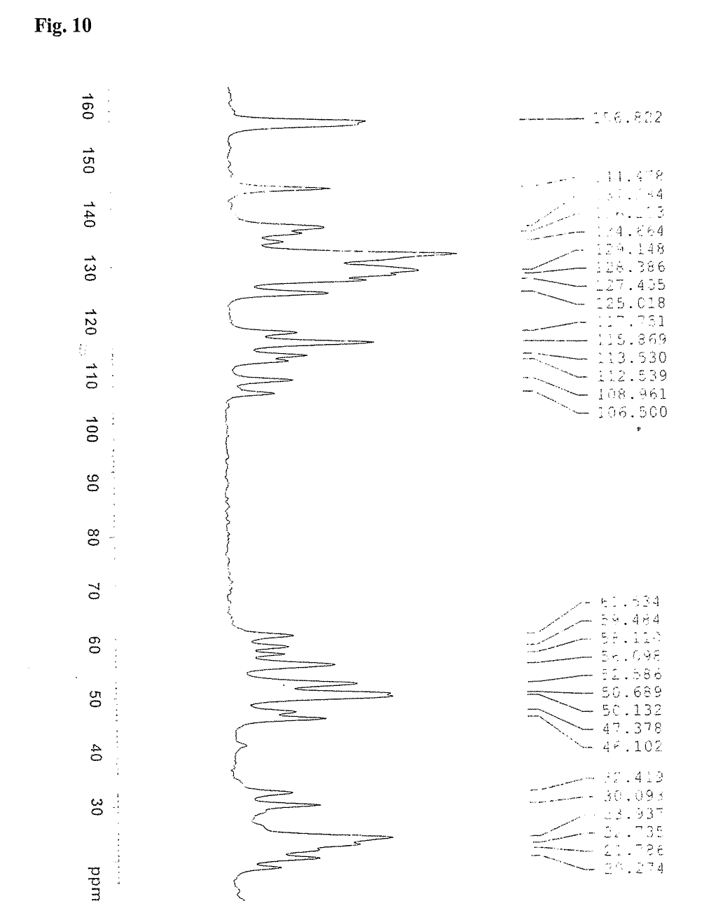

The methoxy derivative 3 38.5 g is refluxed in 400 ml of 48% hydrobromic acid in an argon atmosphere for 5 hours. Then the reaction mixture is left to cool down until the next day. In the reaction mixture slightly pinkish lasofoxifene hydrobromide 2a precipitates. The suspension is poured onto crushed ice and stirred until its dissolution. The solid product is filtered off and washed with water until neutral reaction. The raw product is obtained in the yield of 42.8 g, i.e. 96.2%. The dried, solid, pinkish hydrobromide is stirred under the laboratory temperature in 1300 ml of ethanol. The mixture is stirred and heated to the reflux temperature until dissolution of the solid substance. The solution is filtered while hot. Being stirred, the filtrate is left to cool down to the laboratory temperature spontaneously, whereupon the product precipitates. The mixture is additionally cooled to the temperature of 5-10° C. (refrigerator until the next day). The product is filtered off and dried at the temperature of 105° C. until constant weight. 28 g of the product 2a are obtained with the content of the main substance determined with HPLC to be more than 97%. The melting point of the substance is in the range of 220-222° C. The substance is characterized with FT-IR (KBr), see FIG. 6, FT-Raman, see FIG. 7, DSC, see FIG. 8, X-ray, see FIG. 1 and ¹³C CP-MAS NMR, see FIG. 10. A larger number of signals than the number of carbon atoms indicates a more complex arrangement of more molecules in one cell of the crystal lattice.

### Example 3

### Preparation of cis-6-phenyl-5-[4-(2-pyrrolidin-1-ylethoxy)phenyl]-5,6,7,8-tetrahydronaphthalen-2-ol (2b)

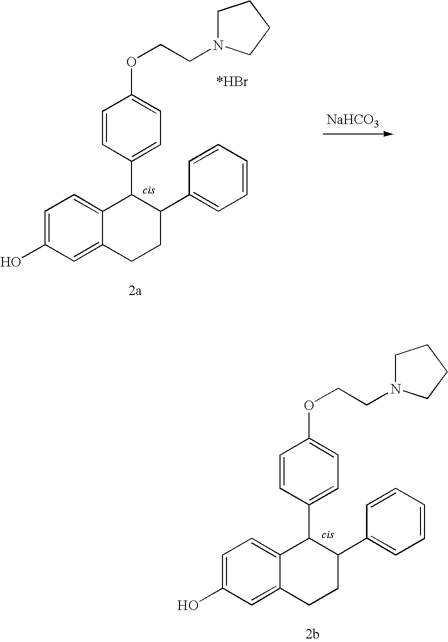

The hydrobromide 2a 26.8 g is dissolved in 270 ml of dichloromethane and 90 ml of methanol at the laboratory temperature and 360 ml of 10% solution of NaHCO₃ are added to the solution. The mixture is stirred vigorously for 1.5 h and then the fractions are separated. The aqueous layer is extracted twice more with a mixture of dichloromethane-methanol in the proportion of 3:1. The organic fractions are put together, dried and the solvents are removed by distillation. This way the base 2b is obtained in the form of solidified, nearly white foam with the yield of ca. 22 g, i.e. 99%.

### Example 4

### Preparation of the crystalline base of lasofoxifene 2b as a Solvate with diethylether

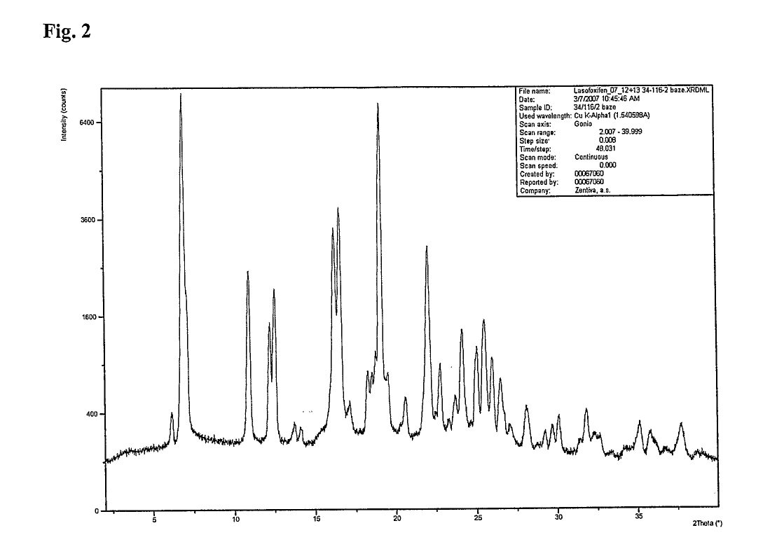

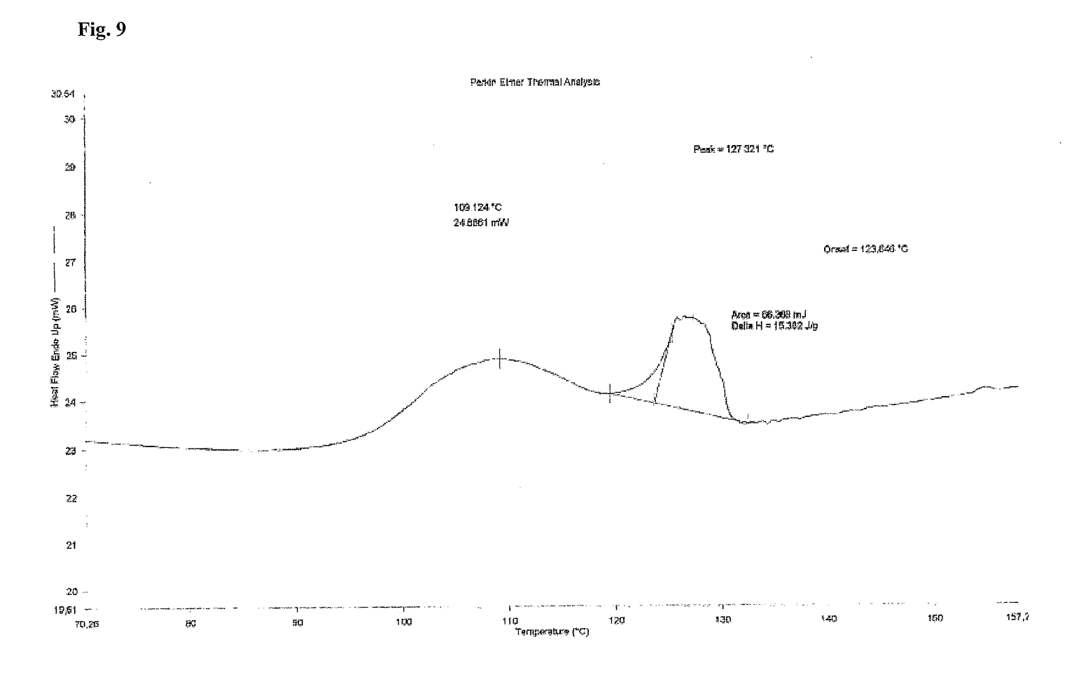

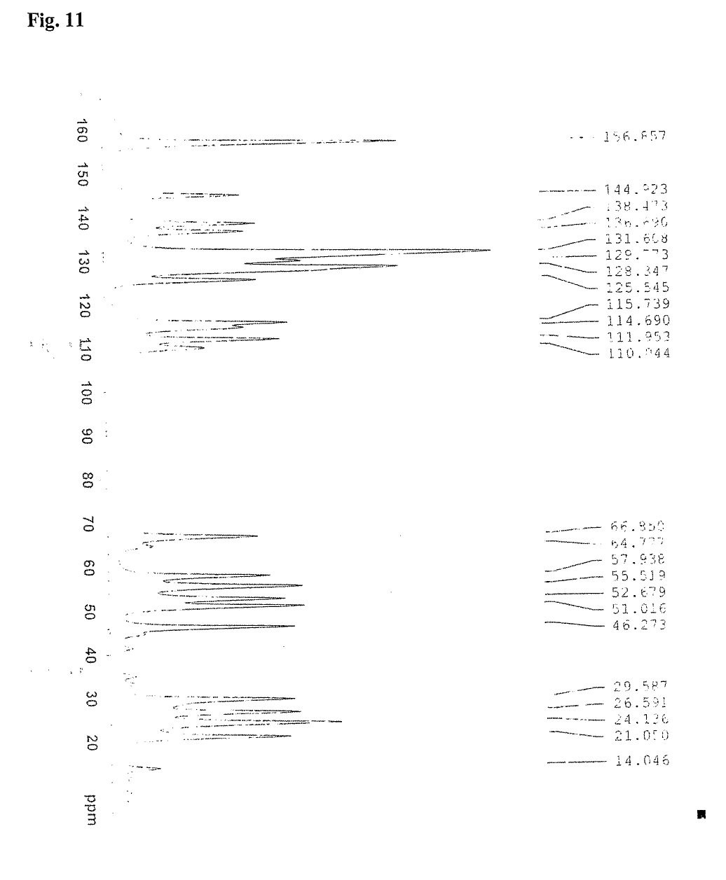

The free base 2b in the quantity of 22.2 g is suspended in 220 ml of diethyl ether and under reflux ca. 600 ml of methanol are added gradually until complete dissolution of the solid substance. While being stirred the solution is left to cool down to the laboratory temperature spontaneously, whereupon and the product is separated. The mixture is additionally cooled to the temperature of 5-10° C. (refrigerator until the next day). The product is filtered off and dried at the temperature of 50° C. until constant weight. This way the crystalline base 2b is obtained in the form of a solvate with diethyl ether polymorph I as a white crystalline substance in the yield of ca. 17.9 g, i.e. 80.6%. The content of the main substance determined by HPLC is more than 99%. The melting point of the substance is in the range of 106-108° C. The substance is characterized with FT-IR (KBr), see FIG. 6, FT-Raman, see FIG. 7, ¹³C CP-MAS NMR, see FIG. 11, DSC, see FIG. 9 and X-ray, see FIG. 2.

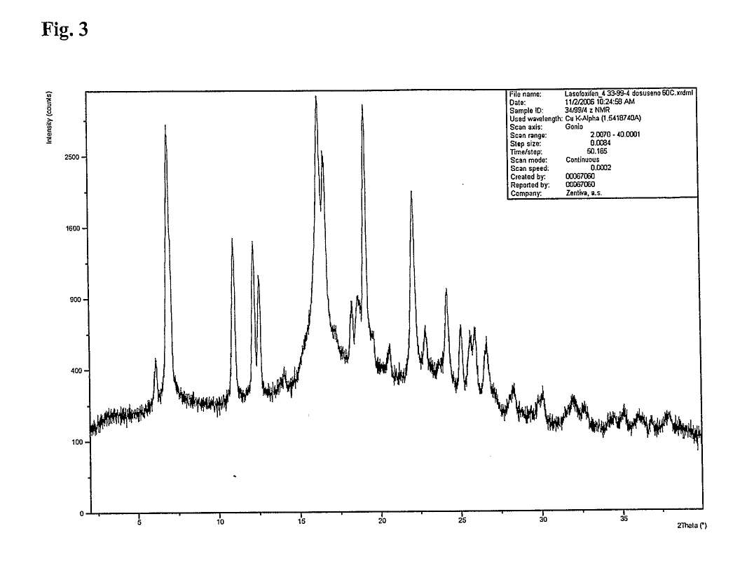

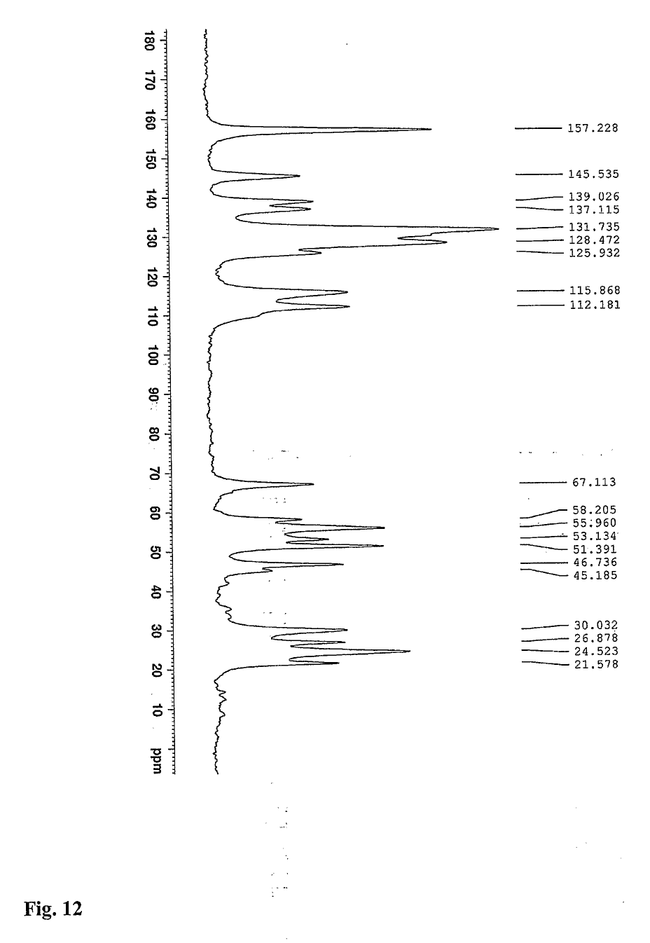

By drying this substance at 60° C. in vacuum one can remove diethyl ether from the crystal lattice in 72-96 h and thus prepare polymorph II without changing the polymorphy of the crystalline base in accordance with X-ray, as illustrated with ¹³C CP-MAS NMR, see FIG. 12 and X-ray, see FIG. 3.

### Example 5

### Crystallization of lasofoxifene base 2b from ethanol

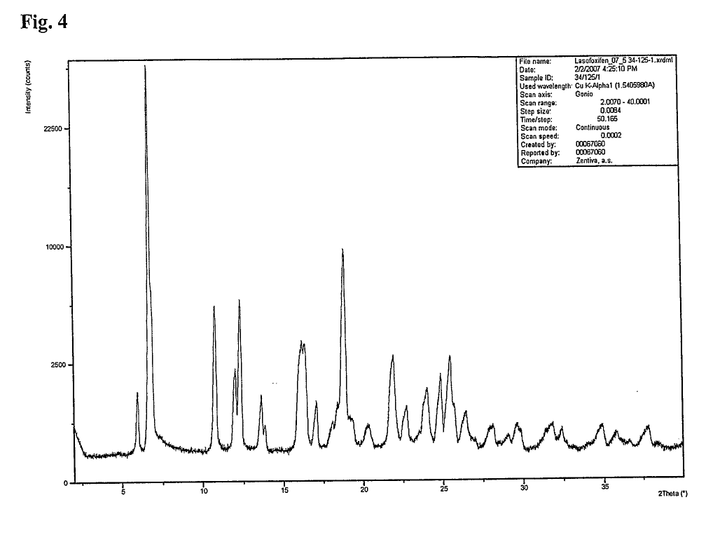

The crystalline solvate of the lasofoxifen base 0.42 g, prepared in accordance with Example 3, is dissolved in 5.5 ml of absolute ethanol under reflux. After spontaneous cooling the product precipitates. The mixture is then cooled to 5-10° C. (refrigerator until the next day). The product is filtered off and dried at the temperature of 50° C. until constant weight. This way the white crystalline base 2b is obtained as a white crystalline substance polymorph II in the yield of ca. 0.32 g, i.e. 76%. The content of the main substance determined with HPLC is more than 99.7%. The melting point of the substance is in the range of 108-112° C. The substance is characterized with X-ray, se FIG. 4.

### Example 6

### Crystallization of lasofoxifene base 2b from acetone

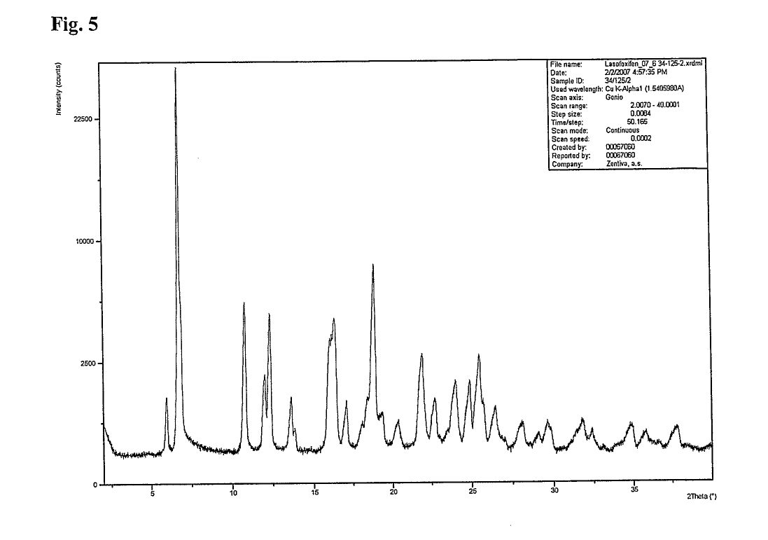

The crystalline solvate of the lasofoxifen base 0.42 g, prepared in accordance with Example 3, is dissolved in 10 ml of acetone under reflux. After spontaneous cooling the product precipitates. The mixture is then cooled to 5-10° C. (refrigerator until the next day). The product is filtered off and dried at the temperature of 50° C. until constant weight. This way the white crystalline base 2b is obtained as a white crystalline substance polymorph II in the yield of ca. 0.34 g, i.e. 79%. The content of the main substance determined with HPLC is more than 99.7%. The melting point of the substance is in the range of 108-112° C. The substance is characterized with X-ray, see FIG. 5.

### Example 7

### Preparation of (−)-cis-(5R,6S)-6-phenyl-5-[4-(2-pyrrolidin-1-ylethoxy)phenyl]-5,6,7,8-tetrahydronaphthalen-2-ol d-tartrate-lasofoxifene

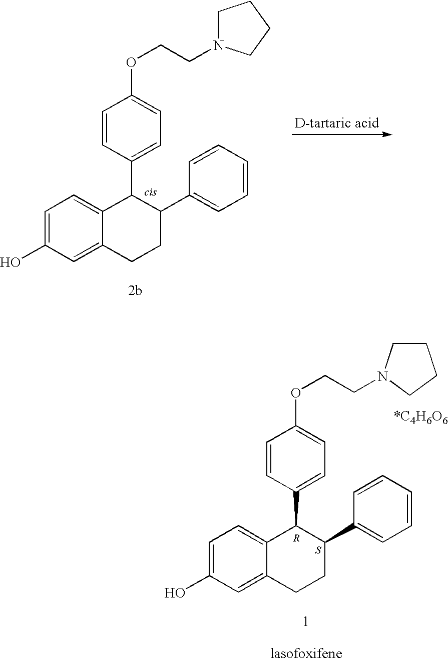

The racemic base 2b, prepared in Example 3, optionally purified as described in Example 4, 5 or 6, in the quantity of 17.2 g is dissolved in 172 ml of warm wine spirit. A solution of 6.24 g of D-tartaric acid in 62 ml of wine spirit is added to this solution and the resulting solution is carefully heated to slight reflux for 5 minutes. During spontaneous cooling the required lasofoxifene D-tartrate precipitates in the yield of 11.8 g, i.e. 50.3%.

1-43. (canceled)

44. A method for the preparation of (−)-cis-(5R,6S)-6-phenyl-5-[4-(2-pyrrolidin-1-ylethoxy)phenyl]-5,6,7,8-tetrahydronaphthalen-2-ol D-tartrate-lasofoxifene of formula (1), comprising the following steps:
a. Preparation of cis-1-[2-[4-(2-phenyl-6-methoxy-1,2,3,4-tetrahydronaphthalen-1-yl)-phenoxy]ethyl]pyrrolidine of formula (3) by alkylation of cis-1-(4-hydroxyphenyl)-2-phenyl-6-methoxy-1,2,3,4-tetrahydronaphthalene with 1-(2-chloroethyl)pyrrolidine base or its salt,
b. Deprotection of the hydroxyl group in the substance of formula (3) in 48% hydrobromic acid at the reflux temperature resulting in crystalline cis-6-phenyl-5-[4-(2-pyrrolidin-1-ylethoxy)phenyl]-5,6,7,8-tetrahydronaphthalen-2-ol hydrobromide of formula (2a),
c. Conversion of the substance of formula (2a) into cis-6-phenyl-5-[4-(2-pyrrolidin-1-ylethoxy)phenyl]-5,6,7,8-tetrahydronaphthalen-2-ol of formula (2b),
d. Preparation of lasofoxifene of formula (1) by conversion into the corresponding diasteroisomer by reaction with D-tartaric acid and crystallization.

45. The method in accordance with claim 44, wherein the deprotection in step (b) is carried out for 2 to 8 hours.

46. The method in accordance with claim 45, wherein the reaction is carried out for 4 to 6 hours.

47. The method in accordance with claim 46, wherein the substance of formula (2a) is crystallized from ethanol.

48. The method in accordance with claim 44, wherein in step (c) the substance of formula (2b) is isolated in crystalline state in the form of a solvate.

49. The method in accordance with claim 48, wherein the substance of formula (2b) is isolated in the form of a solvate with diethyl ether.

50. The method in accordance with claim 49, wherein the proportion of the substance of formula (2b) and diethyl ether in the crystal is 1:0.1 to 0.5.

51. The method in accordance with claim 50, wherein the said solvate is obtained in the crystalline form I, in which the proportion of both the components of the solvate of the substance of formula (2b) to diethyl ether is 1:0.3.

52. The method in accordance with claim 49, wherein it comprises stirring of the substance of formula (2b) in diethyl ether and subsequent addition of methanol under reflux until dissolution of the substance, whereupon after cooling the solvate polymorph I precipitates.

53. The method in accordance with claim 48, wherein the substance of formula (2b) is isolated in the crystalline form II characterized by the following reflections of the powder diffraction pattern: 6.92, 10.90, 12.21, 16.20, 19.13, 24.13, 25.52 (° 2θ) and the melting point of 108 to 112° C.

54. The method in accordance with claim 53, wherein it comprises stirring of the substance of formula (2b) in any of C1-C4 alcohols under reflux and separation of polymorph II after cooling.

55. The method in accordance with claim 54, wherein absolute ethanol is used as the solvent.

56. The method in accordance with claim 53, wherein it comprises stirring of the substance of formula (2b) in acetone.

57. The method in accordance with claim 44, wherein cis-1-{2-[4-(2-phenyl-6-methoxy-1,2,3,4-tetrahydronaphthalen-1-yl)phenoxy]ethyl}pyrrolidine of formula (3) is prepared by refluxing cis-1-(4-hydroxyphenyl)-2-phenyl-6-methoxy-1,2,3,4-tetrahydronaphthalene of formula (33) with 1-(2-chloroethyl)pyrrolidine hydrochloride and an inorganic base in acetone.

58. The method in accordance with claim 57, wherein the reaction is carried out for 15 to 40 hours.

59. The method in accordance with claim 57, wherein the reaction is carried out for 20 to 25 hours.

60. The method in accordance with claim 57, wherein freshly calcined potassium carbonate is used as the base in the reaction.

61. The salt of hydrobromic acid with cis-6-phenyl-5-[4-(2-pyrrolidin-1-ylethoxy)phenyl]-5,6,7,8-tetrahydronaphthalen-2-ol of formula 2a, including its solvates, 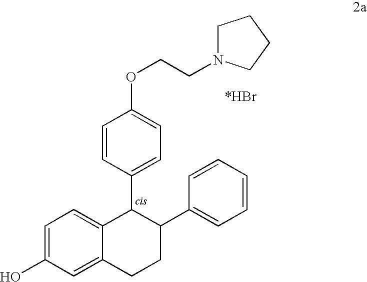
said salt being in a crystalline form and having a purity higher than 97%.

62. The salt in accordance with claim 61, characterized by the following reflections in the X-ray diffraction pattern: 7.01, 8.81, 13.53, 14.52, 15.55, 17.36, 19.13, 21.85, 25.62 (° 2θ).

63. The salt in accordance with claim 61, having its melting point in the temperature interval of 220 to 225° C.

64. The salt in accordance with claim 61, having the DSC Onset 221.5±2.0 and Peak 226.0±2.0° C.

65. The salt in accordance with claim 61, characterized by the following FT-IR (KBr) bands: 3176 (br), 2952, 2835, 2706, 1606, 1505, 1449, 1254, 1240, 1174, 1067, 829, 756, 698 (cm⁻¹).

66. The salt in accordance with claim 61, characterized by the following FT-Raman bands: 3059, 2946, 2866, 1606, 1583, 1439, 1175, 1001, 735 (cm⁻¹).

67. The salt in accordance with claim 61, characterized by the following signals in ¹³C CP-MAS NMR: C—CH₂ 20.27, 21.78, 22.73, 23.93, 30.09, 32.41; CH 46.10, 47.37, 50.13, 50.68; N—CH₂ 52.58, 56.09, 58.11; O—CH₂ 59.48, 61.53; C arom. 106.50, 108.96, 112.53, 113.53, 115.86, 117.75, 125.01, 127.40, 128.38, 129.14, 134.66, 136.25, 137.28, 144.47, 156.82 (ppm).
